(19) United States
(12) Patent Application Publication
(10) Pub. No.: US 2025/0186007 A1
(43) Pub. Date: Jun. 12, 2025

(54) MEDICAL DIAGNOSIS APPARATUS, CONTROL METHOD OF MEDICAL DIAGNOSIS APPARATUS, AND CONTROL PROGRAM OF MEDICAL DIAGNOSIS APPARATUS

(71) Applicant: FUJIFILM Corporation, Tokyo (JP)

(72) Inventor: Ryo YOSHIDA, Tokyo (JP)

(73) Assignee: FUJIFILM Corporation, Tokyo (JP)

(21) Appl. No.: 18/974,804

(22) Filed: Dec. 10, 2024

(30) Foreign Application Priority Data
Dec. 12, 2023 (JP) 2023-209341

(51) Int. Cl. A61B 6/04; A61B 6/00; A61B 6/03

(52) U.S. Cl. CPC A61B 6/0492; A61B 6/032; A61B 6/5258; A61B 6/542

(57) ABSTRACT

The medical diagnosis apparatus includes a processor, in which the processor acquires subject information of a subject placed on a bed, acquires a positioning image based on at least any of an optical image of the subject in a state of being placed on the bed or a scanogram image of the subject, on the basis of the subject information, and projects the acquired positioning image onto the bed or a space above the bed.

### CROSS-REFERENCE TO RELATED APPLICATION

This application claims priority from Japanese Patent Application No. 2023-209341, filed Dec. 12, 2023, the disclosure of which is incorporated herein by reference in its entirety.

### BACKGROUND

### Technical Field

The present disclosure relates to a medical diagnosis apparatus, a control method of the medical diagnosis apparatus, and a control program of the medical diagnosis apparatus.

### 2. DESCRIPTION OF THE RELATED ART

Techniques of an X-ray medical diagnosis apparatus that can set a more preferable scan plan while reducing an X-ray radiation exposure dose of a subject and can simplify an operator's work have been disclosed (for example, refer to JP2007-7225A, JP2014-117368A, and JP7051400B).

In a medical diagnosis apparatus that performs an examination by placing a subject on a bed, time related to positioning of the subject on the bed hinders an examination throughput. In addition, in a medical diagnosis apparatus that performs an examination by placing a subject on a bed, the subject is exposed to radiation in imaging for an imaging plan after positioning the subject on the bed.

### SUMMARY

The present disclosure has been made in view of the above-described points, and an object thereof is to provide a medical diagnosis apparatus, a control method of the medical diagnosis apparatus, and a non-transitory storage medium storing a program of the medical diagnosis apparatus that can reduce a radiation exposure dose of a subject and improve a workflow of an examination as compared with the related art, in the medical diagnosis apparatus that performs an examination by placing the subject on a bed.

A medical diagnosis apparatus according to a first aspect of the present disclosure comprises a processor, in which the processor acquires subject information of a subject placed on a bed, acquires a positioning image based on at least any of an optical image of the subject in a state of being placed on the bed or a scanogram image of the subject, on the basis of the subject information, and projects the acquired positioning image onto the bed or a space above the bed.

In a medical diagnosis apparatus according to a second aspect of the present disclosure, in the medical diagnosis apparatus according to the first aspect, the processor changes the positioning image to be projected onto the bed or the space above the bed before and after the subject is placed on the bed.

In a medical diagnosis apparatus according to a third aspect of the present disclosure, in the medical diagnosis apparatus according to the first aspect, the processor recognizes a color of clothes worn by the subject, and processes the positioning image into a color such that the positioning image is able to be recognized in a case where the positioning image is projected onto the clothes.

In a medical diagnosis apparatus according to a fourth aspect of the present disclosure, in the medical diagnosis apparatus according to the first aspect, the positioning image is the optical image of the subject or the scanogram image of the subject itself.

In a medical diagnosis apparatus according to a fifth aspect of the present disclosure, in the medical diagnosis apparatus according to the first aspect, the positioning image is a reference image generated by cutting out a predetermined reference site from the optical image of the subject or the scanogram image of the subject.

In a medical diagnosis apparatus according to a sixth aspect of the present disclosure, in the medical diagnosis apparatus according to the fifth aspect, the positioning image is an image obtained by superimposing the reference image on the optical image of the subject or the scanogram image of the subject itself.

In a medical diagnosis apparatus according to a seventh aspect of the present disclosure, in the medical diagnosis apparatus according to the first aspect, the processor generates the scanogram image of the subject, and generates a scanogram image of only a reference site corresponding to the reference image.

In a medical diagnosis apparatus according to an eighth aspect of the present disclosure, in the medical diagnosis apparatus according to the first aspect, the processor generates the scanogram image of the subject, and skips the generation of the scanogram image in a case where the positioning image is projected onto the bed or the space above the bed.

In a medical diagnosis apparatus according to a ninth aspect of the present disclosure, in the medical diagnosis apparatus according to the first aspect, the processor determines a rate of match between the subject placed on the bed and the positioning image.

In a medical diagnosis apparatus according to a tenth aspect of the present disclosure, in the medical diagnosis apparatus according to the ninth aspect, the processor performs a notification regarding a placement position of the subject on the bed according to the determined rate of match.

In a medical diagnosis apparatus according to an eleventh aspect of the present disclosure, in the medical diagnosis apparatus according to the tenth aspect, the processor performs a notification of an adjustment amount of the placement position of the subject on the bed.

In a medical diagnosis apparatus according to a twelfth aspect of the present disclosure, in the medical diagnosis apparatus according to the ninth aspect, the processor controls an examination sequence of the subject according to the determined rate of match.

In a medical diagnosis apparatus according to a thirteenth aspect of the present disclosure, in the medical diagnosis apparatus according to the ninth aspect, the processor changes a projection position of the positioning image according to the determined rate of match.

In a medical diagnosis apparatus according to a fourteenth aspect of the present disclosure, in the medical diagnosis apparatus according to the first aspect, in a case where the scanogram image of the subject is used as the positioning image, the processor controls an irradiation dose of X-rays to the subject using dose data of the X-rays in a case of generating the scanogram image.

In a medical diagnosis apparatus according to a fifteenth aspect of the present disclosure, in the medical diagnosis apparatus according to the fourteenth aspect, the processor performs denoising in a case where a noise amount of an examination image of the subject obtained by controlling the irradiation dose of the X-rays to the subject exceeds a predetermined allowable amount.

A control method of a medical diagnosis apparatus according to a sixteenth of the present disclosure comprises, via a processor, acquiring subject information of a subject, acquiring a positioning image based on at least any of an optical image of the subject in a state of being placed on a bed where the subject is placed or a scanogram image of the subject, on the basis of the subject information, and projecting the positioning image onto the bed or a space above the bed.

A non-transitory storage medium storing program of a medical diagnosis apparatus according to a seventeenth aspect of the present disclosure causes a computer to execute processing comprising acquiring subject information of a subject; acquiring a positioning image based on at least any of an optical image of the subject in a state of being placed on a bed where the subject is placed or a scanogram image of the subject, on the basis of the subject information; and projecting the positioning image onto the bed or a space above the bed.

According to the present disclosure, it is possible to provide a medical diagnosis apparatus, a control method of the medical diagnosis apparatus, and a non-transitory storage medium storing a program of the medical diagnosis apparatus that can reduce a radiation exposure dose of a subject and improve a workflow of an examination as compared with the related art, in the medical diagnosis apparatus that performs an examination by placing the subject on a bed.

### BRIEF DESCRIPTION OF THE DRAWINGS

FIG. 1 is a diagram illustrating a schematic configuration of a medical diagnosis apparatus according to an embodiment of the disclosed technology.

FIG. 2 is a block diagram illustrating an example of a functional configuration of a console.

FIG. 3 is a diagram illustrating a state in which a positioning image is projected onto a bed.

FIG. 4 is a diagram illustrating a state in which a positioning image and reference marks are projected onto a bed.

FIG. 5 is a diagram illustrating a state in which a positioning image is projected onto a bed.

FIG. 6 is a diagram illustrating a state in which a positioning image is projected onto a bed.

FIG. 7 is a diagram illustrating a state in which a positioning image and a line indicating a contour are projected onto a bed.

FIG. 8 is a diagram illustrating a state in which a positioning image and a line indicating a contour are projected onto a bed.

FIG. 9 is a flowchart illustrating a flow of control of a medical diagnosis apparatus by a console.

### DESCRIPTION OF THE PREFERRED EMBODIMENTS

Hereinafter, examples of embodiments of the present disclosure will be described with reference to the drawings. Note that the same or equivalent constituents and parts in each drawing are denoted by the same reference numerals. In addition, dimensional ratios in the drawings are exaggerated for convenience of description, and may be different from actual ratios.

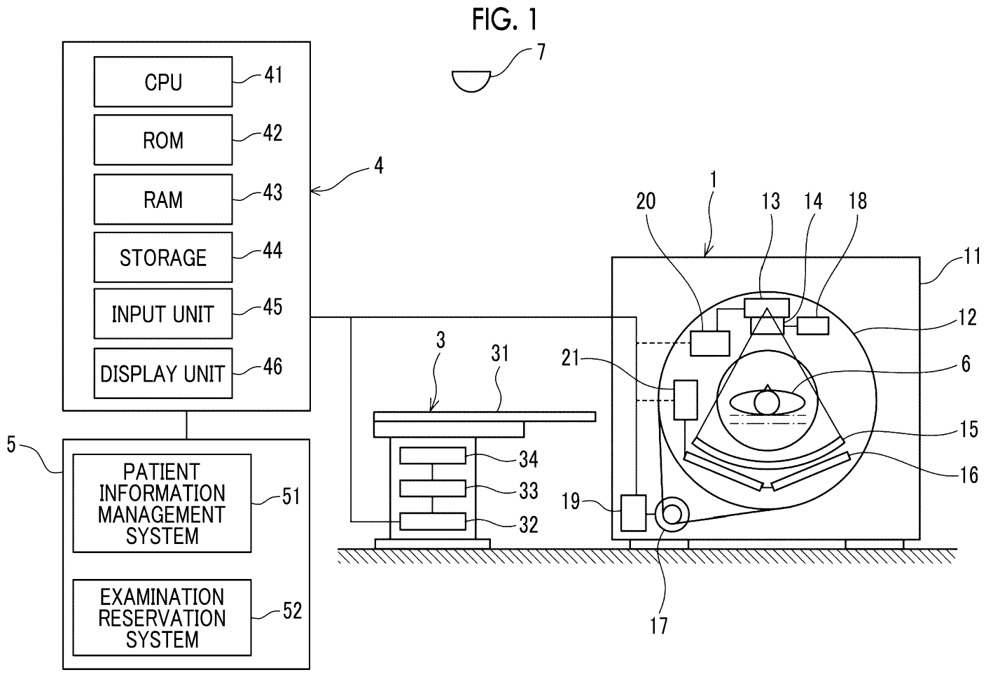

FIG. 1 is a diagram illustrating a schematic configuration of a medical diagnosis apparatus according to the present embodiment. In the present embodiment, a configuration of an embodiment of an X-ray CT apparatus as a medical diagnosis apparatus is illustrated. Note that a medical diagnosis apparatus according to the embodiment of the present disclosure is not limited to the X-ray CT apparatus, and can be applied to other medical diagnosis apparatuses as long as the apparatus is a device in which a bed is moved up and down.

The medical diagnosis apparatus according to the present embodiment includes a scanner 1, a bed 3, and a console 4.

The scanner 1 is a part that executes a CT scan. The scanner 1 includes a stand (gantry) 11, a rotary plate 12 that has an opening in a central portion and is rotatably supported by the stand 11, an X-ray tube device 13 that is fixed to the rotary plate 12, a collimator 14 that is provided in an X-ray radiation port portion of the X-ray tube device 13, an X-ray detector 15 that is disposed to face the X-ray tube device 13 with the opening of the rotary plate 12 interposed therebetween, and a rotary plate drive device 17 that is provided in the stand 11. In addition, the rotary plate 12 of the scanner 1 includes a collimator control device 18 that controls the collimator 14 to change an X-ray irradiation field, a rotary plate drive control device 19 that performs a drive control of the rotary plate drive device 17, an X-ray high-voltage generation device 20 that supplies power for X-ray generation to the X-ray tube device 13 and controls an X-ray generation condition, a data collection device 16 that collects an output of the X-ray detector 15, and a data transmission device 21 that transmits data collected by the data collection device 16. Note that the supply of power and control signals to each unit provided in the rotary plate 12 and the extraction of data from each unit provided in the rotary plate 12 are performed via a slip ring (not illustrated) provided between the stand 11 and the rotary plate 12.

The bed 3 moves the subject between an imaging preparation position and an imaging position. The subject is placed on a top plate 31. There are provided an up-down movement mechanism and a front-rear movement mechanism of the top plate 31 (which are not illustrated). Further, the bed 3 is provided with a bed control device 32, a top plate up-down movement control device 33, and a top plate front-rear movement control device 34 in order to control the operation of the up-down movement mechanism and the front-rear movement mechanism of the top plate 31.

The console 4 controls a medical diagnosis apparatus that is an X-ray CT apparatus. The console 4 is an example of a computer according to the embodiment of the present disclosure. The console 4 includes a central processing unit (CPU) 41, a read only memory (ROM) 42, a random access memory (RAM) 43, and a storage 44. In addition, the console 4 may further include an input unit 45 and a display unit 46.

The CPU 41 as an example of a processor is a central arithmetic processing unit, and executes various programs or controls each unit. That is, the CPU 41 reads out the program from the ROM 42 or the storage 44, and executes the program using the RAM 43 as a work area. The CPU 41 performs control and various kinds of arithmetic processing of the above configurations in accordance with the program recorded in the ROM 42 or the storage 44. In the present embodiment, the ROM 42 or the storage 44 stores a control program of the medical diagnosis apparatus that projects a positioning image onto the bed 3 of the medical diagnosis apparatus and controls the operation of the medical diagnosis apparatus.

The positioning image is an image based on at least any of an optical image of the subject in a state of being placed on the bed 3 or a scanogram image of the subject. The positioning image may be an optical image of the subject or a scanogram image of the subject itself, or may be a reference image generated by cutting out a predetermined reference site from the optical image of the subject or the scanogram image of the subject. The reference site refers to, for example, a site of the shoulder area, a site of the pelvis area, or the like, which is a reference for positioning of the subject in a case where the subject is placed on the bed 3. Note that the reference image may be an image of the entire site to be a landmark such as a shoulder or a waist of a person, may be a partial image useful for positioning, such as a contour or a tip part of the site, or may be a line extracted from the contour or the tip part of the site. The optical image of the subject may be captured by one or a plurality of imaging apparatuses provided in the ceiling or the wall surface of an examination room, the periphery of the stand 11, and the like.

In addition, the positioning image may be an image obtained by superimposing the above-described reference image on the optical image of the subject or the scanogram image of the subject.

The ROM 42 stores various programs and various kinds of data. The RAM 43 as a work area temporarily stores the program or the data. The storage 44 is configured with a storage device such as a hard disk drive (HDD), a solid state drive (SSD), or a flash memory, and stores various kinds of data and various programs including an operating system.

The input unit 45 includes a pointing device, such as a mouse, and a keyboard, and is used to perform various inputs. The input unit 45 includes a bed operator that operates a height of the bed 3.

The display unit 46 is, for example, a liquid crystal display, and displays various kinds of information. The display unit 46 may be configured to function as the input unit 45 by adopting a touch panel method.

In the present embodiment, the console 4 is connected to a hospital information system 5 of a hospital in which the X-ray CT apparatus is installed. The hospital information system 5 includes a patient information management system 51, and an examination reservation system 52.

The patient information management system 51 is a database in which personal information, medical data, examination image data, examination data, medication data, and the like of a patient who has received medical care in the hospital in the past are stored. The personal information of the patient includes physical information such as height and weight, and a past medical examination date, in addition to the personal specification information such as a name, an ID, a date of birth, an age, and a gender. These pieces of personal information are input to the system in a case where the subject visits a hospital as a patient and receives medical care. Note that the height and weight of the patient are collected by the patient's self-reporting on a questionnaire or by actual measurement, and are input to the patient information management system 51. In addition, the medical data, the examination image data, the examination data, and the medication data are input to the patient information management system for each time of the medical care of a patient, and an in-hospital patient database is created.

The examination reservation system 52 creates an examination schedule on that day for each system of the medical diagnosis apparatus provided in the hospital on the basis of the information stored or written in the patient information management system, and distributes the created examination schedule as data to each system. The examination schedule includes an order in which patients will be examined that day in the medical diagnosis apparatus, and personal specification information and physical information of each patient. Note that the examination reservation system 52 sequentially updates the content of the examination in accordance with the progress of the medical care of that day, and transmits the updated data to each system each time the data is updated.

The console 4 projects the positioning image from a projection device 7 onto the bed 3 or an upper portion of the bed 3. The console 4 projects the positioning image from the projection device 7 onto the bed 3 or the upper portion of the bed 3, and thereby, it is possible to smoothly place the subject on the bed 3 and improve the examination throughput of the subject as compared with a case where the positioning image is not projected. Note that the projection device 7 may be a projector that projects an image, a display device that displays a positioning image for augmented reality (AR) onto the bed 3 or the upper portion of the bed 3, or a virtual reality (VR) goggle that is used by a technician. The projector may be installed on a ceiling or a wall surface of an imaging room where the medical diagnosis apparatus is provided, an inside of the stand 11, an outer surface of the stand 11, or the like. In addition, the number of projectors is not limited to one, and a plurality of projectors may be installed.

In a case where the control program of the medical diagnosis apparatus described above is executed, the console 4 realizes various functions by using the hardware resources. The functional configuration realized by the console 4 will be described.

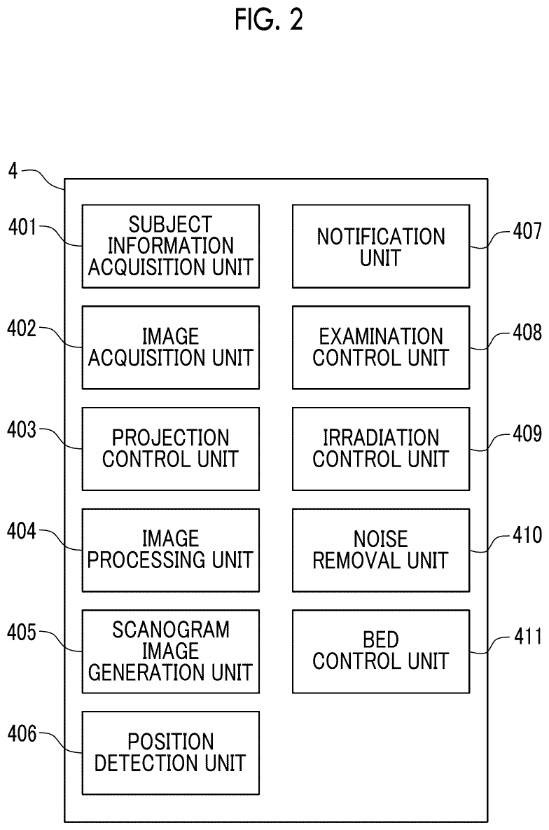

FIG. 2 is a block diagram illustrating an example of the functional configuration of the console 4.

As illustrated in FIG. 2, the console 4 has, as a functional configuration, a subject information acquisition unit 401, an image acquisition unit 402, a projection control unit 403, an image processing unit 404, a scanogram image generation unit 405, a position detection unit 406, a notification unit 407, an examination control unit 408, an irradiation control unit 409, a noise removal unit 410, and a bed control unit 411. Each functional configuration is realized by the CPU 41 reading out and executing a control program of the medical diagnosis apparatus stored in the ROM 42 or the storage 44.

The subject information acquisition unit 401 acquires information regarding the subject who is examined by the medical diagnosis apparatus. The subject information acquisition unit 401 acquires the information regarding the subject, for example, from the patient information management system 51. The subject information acquisition unit 401 acquires the information regarding the subject by performing image recognition on the optical image of the subject captured by the imaging apparatus, for example. In this case, the image of the subject registered in advance may be registered in advance in any place, for example, in the patient information management system 51. The subject information acquisition unit 401 acquires the information regarding the subject by recognizing voice collected by a microphone, for example. In this case, the voice of the subject registered in advance may be registered in advance in any place, for example, in the patient information management system 51. The subject information acquisition unit 401 may acquire the information regarding the subject by reading, for example, a barcode or an IC tag for patient identification used in a hospital or the like.

The image acquisition unit 402 acquires the positioning image on the basis of the subject information acquired by the subject information acquisition unit 401. A storage location of the positioning image may be a recording medium provided inside the console 4, an external recording device connected to the console 4, or the patient information management system 51 connected to the console 4 via a network. Note that in a case where the subject is examined for the first time, the positioning image is not present. Therefore, the image acquisition unit 402 does not acquire the positioning image.

Note that in the case of the subject who is examined for the first time, the image acquisition unit 402 may acquire the positioning image of a subject having a similar body shape to the subject by searching the patient information management system 51. For example, the image acquisition unit 402 may acquire the positioning image of another subject whose height is within a predetermined difference, weight is within a predetermined difference, or waist circumference is within a predetermined difference from the subject who is examined for the first time.

The projection control unit 403 performs control of projecting the positioning image acquired by the image acquisition unit 402 onto the top plate 31 of the bed 3 or a space above the bed 3. The projection of the positioning image may be performed by the projector that projects the image, may be performed by the display device that displays the positioning image on the top plate 31 of the bed 3 or the upper portion of the bed 3 by AR, or may be performed by the VR goggles used by the technician. Note that the projection to the space above the bed 3 may also include the projection to the clothes of the subject placed on the bed 3.

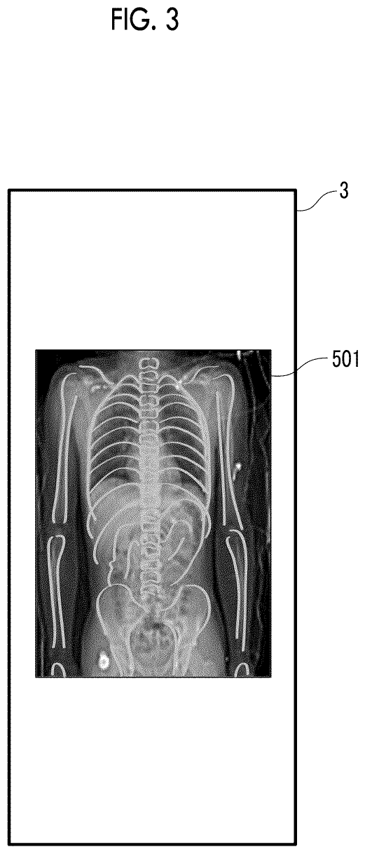

FIG. 3 is a diagram illustrating a state in which a positioning image 501 is projected onto the bed 3. FIG. 3 illustrates a state where a scanogram image acquired from the subject information is projected onto the bed 3 as the positioning image 501.

Note that the projection control unit 403 may change a projection method in accordance with the site of the subject to be aligned. For example, in a case where the site to be aligned is the head, since the rotation direction or the inclination of the head is also important, the projection control unit 403 may perform the projection in two directions or may perform the projection in a three-dimensional manner. In a case where the site to be aligned is the head, the positioning image may be an image of the eyes, the nose, the cars, the top of the head, or the like. In addition, for example, in a case where the site to be aligned is the abdomen, the positioning image may be an image of a portion of the waistline or a protruding portion of the pelvis of the subject. In addition, for example, in a case where the site to be aligned is the lower limb, the positioning image may include a crotch portion, a knee portion, and a foot portion.

The positioning image projected onto the bed 3 is easy to see before the subject is placed on the bed 3, but the positioning image is difficult to see after the subject is placed on the bed 3. Therefore, the projection control unit 403 may change the positioning image projected to the bed 3 or the upper portion of the bed 3 before and after the subject is placed on the bed 3. For example, the projection control unit 403 may perform a control of projecting a normal plane image before the subject is placed on the bed 3, and of projecting a three-dimensional image, projecting the three-dimensional image to a space on a body surface of the subject in consideration of the body thickness of the subject, or projecting the three-dimensional image to clothes of the subject after the subject is placed on the bed 3.

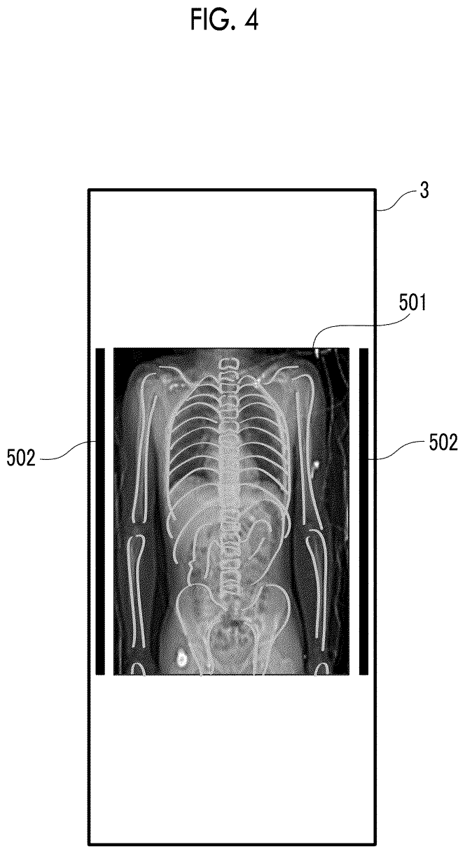

In addition, the projection control unit 403 may perform control of further projecting the reference mark to a position that is not hidden by the subject in order to avoid the positioning image from being hidden by the subject and to make the positioning image difficult to be seen. FIG. 4 is a diagram illustrating a state in which the positioning image 501 and reference marks 502 are projected onto the bed 3. FIG. 4 illustrates a state in which the reference marks 502 are projected to match the vertical length of the positioning image 501.

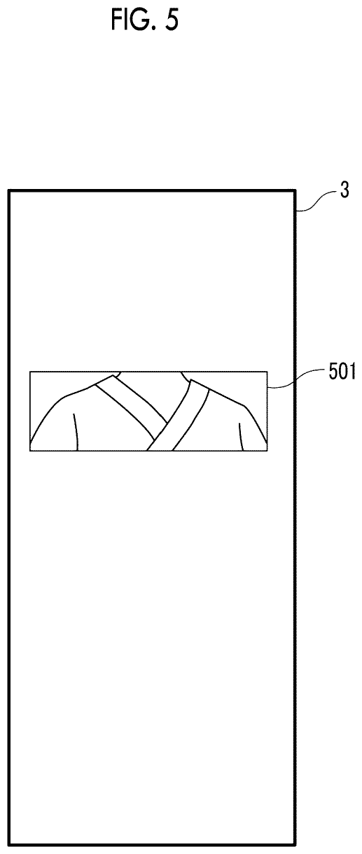

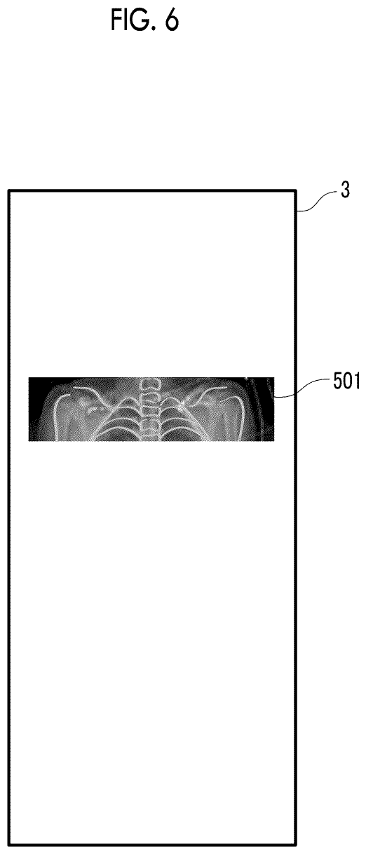

The projection control unit 403 may project, as the positioning image, an image obtained by cutting out a site of a person to be a landmark, such as a shoulder or a waist. FIGS. 5 and 6 are diagrams illustrating a state in which the positioning image 501 is projected onto the bed 3. FIG. 5 illustrates a state where an optical image obtained by cutting out a shoulder portion of the subject is projected as the positioning image 501. FIG. 6 illustrates a state in which a scanogram image obtained by cutting out a shoulder portion of the subject is projected as the positioning image 501. Note that the projection control unit 403 may project the reference marks 502 illustrated in FIG. 4 in addition to the image.

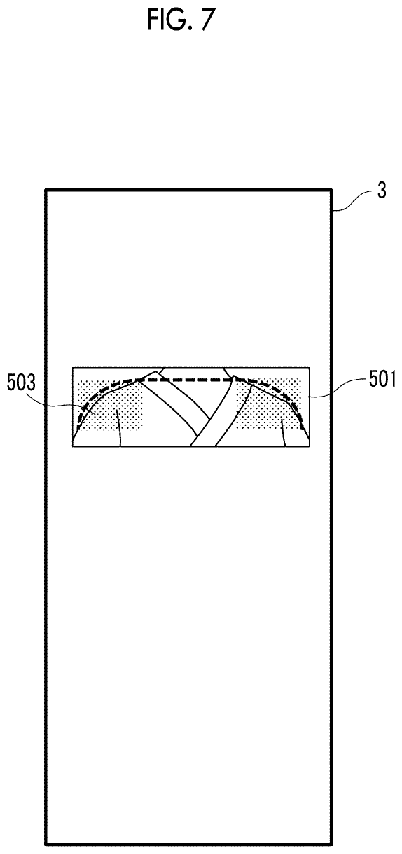

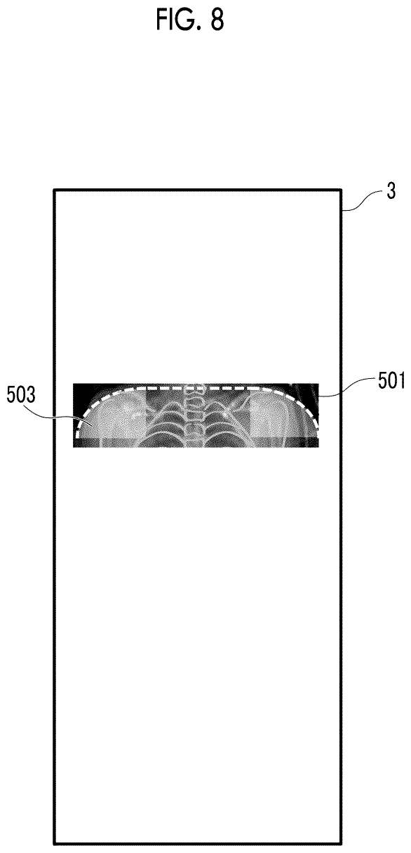

The projection control unit 403 may project a line indicating a contour of a site to be a landmark, such as a shoulder or a waist of a person, to an image obtained by cutting out the site, as the positioning image. FIGS. 7 and 8 are diagrams illustrating a state in which the positioning image 501 and a line 503 indicating the contour are projected onto the bed 3. FIG. 7 illustrates a state where an optical image obtained by cutting out a shoulder portion of the subject and the line 503 indicating the contour of the shoulder are projected as the positioning image 501. FIG. 8 illustrates a state where a scanogram image obtained by cutting out a shoulder portion of the subject and the line 503 indicating the contour of the shoulder are projected as the positioning image 501. Note that the projection control unit 403 may project the reference marks 502 illustrated in FIG. 4 in addition to the image.

The image processing unit 404 performs image processing on the positioning image acquired by the image acquisition unit 402. For example, the image processing unit 404 performs processing of recognizing a color of the clothes worn by the subject and of processing the positioning image into a color such that the positioning image can be recognized in a case where the positioning image is projected onto the clothes. For example, in a case where the color of the clothes worn by the subject is blue, the image processing unit 404 may perform processing of converting the positioning image into a red color.

The scanogram image generation unit 405 executes processing of generating a scanogram image of the subject on the basis of the irradiation of the subject with the X-rays controlled by the irradiation control unit 409, which will be described later. The scanogram image can be obtained by detecting the X-rays transmitted through the subject with the X-ray detector 15. In a case where the reference image is used as the positioning image, the scanogram image generation unit 405 may generate a scanogram image of only the reference site corresponding to the reference image. For example, in a case where the reference site is the shoulder area, the scanogram image generation unit 405 executes processing of generating the scanogram image of the subject on the basis of the X-rays emitted only for the shoulder area. By generating the scanogram image of only the reference site corresponding to the reference image, the medical diagnosis apparatus can suppress the radiation exposure dose of the subject.

The scanogram image generation unit 405 may execute processing of generating a scanogram image for a site different from the site that is a target of the main imaging. For example, in a case where the site as the target of the main imaging is the chest and abdomen, the scanogram image generation unit 405 may execute processing of generating the scanogram image by narrowing down to only the shoulder. By generating the scanogram image for a site different from the site as the target of the main imaging, the medical diagnosis apparatus can reduce the radiation exposure dose of the subject as compared with a case of generating the scanogram image including the site as the target of the main imaging.

In addition, in a case where the positioning image is projected onto the bed 3 or a space above the bed 3, the scanogram image generation unit 405 may perform control to skip the generation of the scanogram image. By skipping the generation of the scanogram image, the medical diagnosis apparatus can reduce the radiation exposure dose of the subject as compared with a case of generating the scanogram image.

The position detection unit 406 determines whether or not the subject is correctly placed on the bed 3 by determining a rate of match between the subject placed on the bed 3 and the positioning image projected by the projection control unit 403. For example, the position detection unit 406 may determine the degree of match by obtaining a degree of overlap between a contour portion of the site of the body in the positioning image and a contour portion of the same site of the subject placed on the bed 3. Specifically, the position detection unit 406 may determine that the subject is not correctly placed on the bed 3 in a case where the ratio of the area in which the two contour portions overlap is less than 80%, and may determine that the subject is correctly placed on the bed 3 in a case where the ratio is 80% or more. In addition, the position detection unit 406 may determine the degree of match by obtaining, for example, an interval between a tip of the contour portion of the site of the body in the positioning image and a tip of the contour portion of the same site of the subject placed on the bed 3. Specifically, the position detection unit 406 may determine that the subject is not correctly placed on the bed 3 in a case where the two tips are separated from each other by 10 mm or more, and may determine that the subject is correctly placed on the bed 3 in a case where the interval between the tips is less than 10 mm. Note that it is needless to say that the above-mentioned numerical values are merely examples, and the present disclosure is not limited to the examples.

In a case where the subject is a severely ill patient, an elderly person, a person with physical disabilities, or the like, even in a case where the subject lies down at a position shifted from the position of the projected positioning image, it may be difficult to change the position of the subject, or the subject may suffer from pain due to the movement. Therefore, the projection control unit 403 may control the projection position of the positioning image projected onto the bed 3 on the basis of a detection result of a shift amount by the position detection unit 406. For example, in a case where the position detection unit 406 detects that the position of the subject is shifted downward by 50 mm and rightward by 30 mm from the projection position of the positioning image, the projection control unit 403 may project the positioning image by shifting the positioning image downward by 50 mm and rightward by 30 mm. In addition, in this case, a shift amount and a shift direction of the projection of the positioning image may be applied to the irradiation control by the irradiation control unit 409 to be described later.

The notification unit 407 performs a notification regarding a placement position of the subject on the bed 3 on the basis of the ratio of match determined by the position detection unit 406. The notification unit 407 may perform the notification regarding the placement position by using the projection display on the bed 3, by using the display on an operation panel or the like, or by using a voice announcement. In addition, in a case where the technician wears the VR goggles, the notification unit 407 may perform the notification regarding the placement position by displaying the notification on the VR goggles worn by the technician.

Specifically, in a case where the position detection unit 406 determines that the subject is not correctly placed on the bed 3, the notification unit 407 may notify that “the positioning is insufficient” or the like regarding the placement position of the subject on the bed 3, and in a case where the position detection unit 406 determines that the subject is correctly placed on the bed 3, the notification unit 407 may notify that “the positioning is appropriate” or the like regarding the placement position of the subject on the bed 3.

The notification unit 407 may perform a notification regarding an adjustment amount of the placement position of the subject on the bed 3. Specifically, in a case where the position detection unit 406 determines that the subject is not correctly placed on the bed 3, the notification unit 407 may perform the notification regarding the adjustment amount of the placement position of the subject on the bed 3. For example, the notification unit 407 may notify that “The patient will be positioned at a good positioning position in a case of moving the patient to the right by 28 mm with the tip of the shoulder as a reference” or the like regarding the adjustment amount of the placement position of the subject on the bed 3. In addition, for example, the notification unit 407 may notify that “The patient is shifted upward by 17 mm from the reference position. Please move the patient downward by 17 mm with the tip of the shoulder as the reference.” or the like regarding the adjustment amount of the placement position of the subject on the bed 3.

The examination control unit 408 controls an examination sequence of the subject by the medical diagnosis apparatus. For example, the examination control unit 408 controls the examination sequence of the subject by the medical diagnosis apparatus on the basis of the degree of match determined by the position detection unit 406. For example, in a case where the position detection unit 406 determines that the subject is correctly placed on the bed 3, the examination control unit 408 may simplify the imaging of the scanogram image before the examination or may skip the imaging of the scanogram image before the examination, as the control of the examination sequence.

The irradiation control unit 409 controls an irradiation dose of the X-rays from the X-ray tube device 13 to the subject. For example, a large dose is required for the shoulder, a small dose may be required for the lung because the lung is filled with air and thus the attenuation is small, and a large dose is required for the liver because the attenuation is large. The irradiation control unit 409 controls an irradiation dose of the X-rays to the subject in accordance with the site of the subject. In the present embodiment, in a case where the scanogram image of the subject is used as the positioning image, the irradiation control unit 409 controls the irradiation dose of the X-rays to the subject on the basis of dose data of the X-rays in a case of generating the scanogram image. The scanogram image and the image (image of main imaging) obtained in the examination have different required image quality levels, and the image of the main imaging is required to have higher image quality than the scanogram image. Therefore, the irradiation control unit 409 may obtain the irradiation dose of the X-rays to the subject by multiplying the dose data of the X-rays in a case of generating the scanogram image by a coefficient or performing table conversion. The irradiation control unit 409 can obtain an examination image of the subject with a noise amount desired by the operator by controlling the irradiation dose of the X-rays to the subject using the dose data of the X-rays in a case of generating the scanogram image.

In a case where the noise amount of the examination image obtained by irradiating the subject with the X-rays exceeds an allowable amount with respect to the desired noise amount, the noise removal unit 410 performs noise removal (denoising) processing on the examination image. For example, in a case where a desired image quality index (for example, the image noise) is not obtained by the dose modulation based on the dose data of the subject associated with the past scanogram image due to an increase in the weight of the subject, and the image noise is increased, the noise removal unit 410 suppresses the image noise by increasing a strength level of the denoising processing, and executes the automatic reconstruction such that the desired image quality index is obtained to the same extent as in the case of the past examination.

The bed control unit 411 controls the height of the bed 3. The bed control unit 411 may control the height of the bed 3 on the basis of the operation by the technician, or may control the height of the bed 3 on the basis of data at the time of the most recent examination of the subject. The data at the time of the most recent examination of the subject is recorded in, for example, the patient information management system 51, and may be acquired by the subject information acquisition unit 401.

Next, the operation of the console 4 will be described.

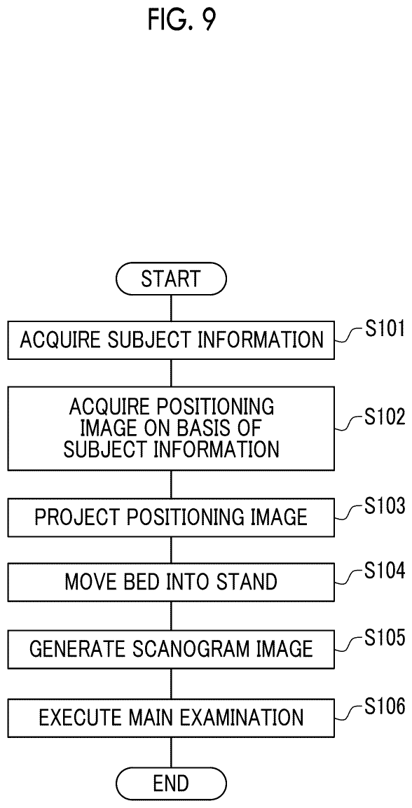

FIG. 9 is a flowchart illustrating a flow of the control of the medical diagnosis apparatus by the console 4. The CPU 41 reads out the control program of the medical diagnosis apparatus from the ROM 42 or the storage 44, and expands and executes the control program on the RAM 43, so that the control processing of the medical diagnosis apparatus is performed. The flowchart illustrated in FIG. 9 is executed in a case where the subject is examined by the medical diagnosis apparatus.

In step S101, the CPU 41 acquires the subject information. The CPU 41 acquires the subject information from, for example, the patient information management system 51.

Following step S101, in step S102, the CPU 41 acquires the positioning image of the subject on the basis of the acquired subject information. Note that in a case where the subject is examined for the first time, since there is no positioning image, the CPU 41 may skip all the subsequent processing related to the positioning image. Note that in the case of the subject who is examined for the first time, the CPU 41 may acquire the positioning image of a subject having a similar body shape to the subject by searching the patient information management system 51.

Following step S102, in step S103, the CPU 41 projects the acquired positioning image onto the bed 3.

In a case where the subject is placed on the bed 3 to match the projected positioning image, the CPU 41 moves the bed 3 into the stand 11 in step S104 following step S103.

Following step S104, in step S105, the CPU 41 generates the scanogram image of the subject by irradiating the subject with the X-rays. Note that in a case where the subject is placed on the bed 3 to match the projected positioning image, the CPU 41 may skip the processing of generating the scanogram image of the subject, or may generate the scanogram image of the subject only for a site different from a site in the case of the main imaging described below.

Following step S105, in step S106, the CPU 41 emits the X-rays to execute a main examination of the subject.

As described above, according to the embodiment of the present disclosure, in the medical diagnosis apparatus that performs the examination by placing the subject on the bed, it is possible to improve the workflow of the examination as compared with the related art. In addition, according to the embodiment of the present disclosure, in the medical diagnosis apparatus that performs the examination by placing the subject on the bed, it is possible to reduce the radiation exposure dose to the subject as compared with the related art.

The embodiments of the present disclosure have been described in detail with reference to the accompanying drawings, but the technical scope of the present disclosure is not limited to such examples. It is apparent that a person having ordinary knowledge in the technical field of the present disclosure can conceive of various modification examples and correction examples within the scope of the technical idea described in the claims, and it is naturally understood that these modification examples and correction examples also belong to the technical scope of the present disclosure.

In addition, the effects described in the embodiment described above are descriptive or exemplary, and are not limited to those described in the embodiment described above. That is, the technique according to the present disclosure can exhibit other effects that are obvious to a person having ordinary knowledge in the technical field of the present disclosure from the description in the embodiment described above, in addition to the effects described in the embodiment described above or instead of the effects described in the embodiment described above.

Note that various processors other than the CPU may execute the control processing of the medical diagnosis apparatus executed by the CPU reading the software (program) in each embodiment described above. Examples of the processors in this case include a programmable logic device (PLD) of which a circuit configuration can be changed after manufacture, such as a field-programmable gate array (FPGA), and a dedicated electric circuit that is a processor having a circuit configuration dedicatedly designed to execute specific processing, such as an application specific integrated circuit (ASIC). In addition, the control processing of the medical diagnosis apparatus may be executed by one of the various processors, or executed by a combination of the same or different kinds of two or more processors (for example, combination of a plurality of FPGAs, combination of the CPU and the FPGA, or the like). In addition, the hardware structures of the various processors are more specifically electrical circuitry where circuit elements such as semiconductor elements are combined.

In addition, in each embodiment described above, an aspect has been described in which the control program of the medical diagnosis apparatus is stored (installed) in advance in the ROM or the storage, but the present disclosure is not limited thereto. The program may be provided by being recorded in a non-transitory recording medium such as a compact disk read only memory (CD-ROM), a digital versatile disk read only memory (DVD-ROM), and a Universal Serial Bus (USB) memory. In addition, the program may be provided by being downloaded from an external device via a network.

What is claimed is:

1. A medical diagnosis apparatus comprising:
a processor,
wherein the processor
acquires subject information of a subject placed on a bed,
acquires a positioning image based on at least any of an optical image of the subject in a state of being placed on the bed or a scanogram image of the subject, on the basis of the subject information, and
projects the acquired positioning image onto the bed or a space above the bed.

2. The medical diagnosis apparatus according to claim 1,
wherein the processor changes the positioning image to be projected onto the bed or the space above the bed before and after the subject is placed on the bed.

3. The medical diagnosis apparatus according to claim 1,
wherein the processor recognizes a color of clothes worn by the subject, and processes the positioning image into a color such that the positioning image is able to be recognized in a case where the positioning image is projected onto the clothes.

4. The medical diagnosis apparatus according to claim 1,
wherein the positioning image is the optical image of the subject or the scanogram image of the subject itself.

5. The medical diagnosis apparatus according to claim 1,
wherein the positioning image is a reference image generated by cutting out a predetermined reference site from the optical image of the subject or the scanogram image of the subject.

6. The medical diagnosis apparatus according to claim 5,
wherein the positioning image is an image obtained by superimposing the reference image on the optical image of the subject or the scanogram image of the subject itself.

7. The medical diagnosis apparatus according to claim 5,
wherein the processor
generates the scanogram image of the subject, and
generates a scanogram image of only a reference site corresponding to the reference image.

8. The medical diagnosis apparatus according to claim 1,
wherein the processor
generates the scanogram image of the subject, and
skips the generation of the scanogram image in a case where the positioning image is projected onto the bed or the space above the bed.

9. The medical diagnosis apparatus according to claim 1,
wherein the processor determines a rate of match between the subject placed on the bed and the positioning image.

10. The medical diagnosis apparatus according to claim 9,
wherein the processor performs a notification regarding a placement position of the subject on the bed according to the determined rate of match.

11. The medical diagnosis apparatus according to claim 10,
wherein the processor performs a notification of an adjustment amount of the placement position of the subject on the bed.

12. The medical diagnosis apparatus according to claim 9,
wherein the processor controls an examination sequence of the subject according to the determined rate of match.

13. The medical diagnosis apparatus according to claim 9,
wherein the processor changes a projection position of the positioning image according to the determined rate of match.

14. The medical diagnosis apparatus according to claim 1,
wherein in a case where the scanogram image of the subject is used as the positioning image, the processor controls an irradiation dose of X-rays to the subject using dose data of the X-rays in a case of generating the scanogram image.

15. The medical diagnosis apparatus according to claim 14,
wherein the processor performs denoising in a case where a noise amount of an examination image of the subject obtained by controlling the irradiation dose of the X-rays to the subject exceeds a predetermined allowable amount.

16. A control method of a medical diagnosis apparatus, the control method comprising:
via a processor,
acquiring subject information of a subject,
acquiring a positioning image based on at least any of an optical image of the subject in a state of being placed on a bed where the subject is placed or a scanogram image of the subject, on the basis of the subject information, and
projecting the positioning image onto the bed or a space above the bed.

17. A non-transitory storage medium storing a program of a medical diagnosis apparatus, the program causing a computer to execute processing comprising:
acquiring subject information of a subject;
acquiring a positioning image based on at least any of an optical image of the subject in a state of being placed on a bed where the subject is placed or a scanogram image of the subject, on the basis of the subject information; and
projecting the positioning image onto the bed or a space above the bed.
